(19) United States
(12) Patent Application Publication
(10) Pub. No.: US 2026/0272635 A1
(43) Pub. Date: Sep. 17, 2026

(54) MEDICAL DEVICE FOR TREATING ANORECTAL DISORDERS

(71) Applicant: ANACURE MEDICAL LTD, Petach-Tikva (IL)

(72) Inventors: Eli MICHAELI, Petach-Tikva (IL); Michael MASTAI, Ramat-Gan (IL)

(21) Appl. No.: 19/167,970

(22) PCT Filed: Mar. 21, 2024
(86) PCT No.: PCT/IL2024/050295

§ 371 Date: Sep. 23, 2025

Related U.S. Application Data
(60) Provisional application No. 63/454,073, filed on Mar. 23, 2023.

(51) Int. Cl. A61F 2/00; A61F 5/00

(52) U.S. Cl. CPC A61F 2/0009; A61F 5/0093; A61F 2220/0008; A61F 2250/0067

(57) ABSTRACT

Novel devices to be positioned in the lumen of a hollow organ or a part thereof, comprising a mesh that allows anchoring of the tubular sleeve to the surface of the lumen of the hollow organ by becoming physically engaged with the surface of the lumen of the hollow organ are provided.

### FIELD OF THE INVENTION

The invention relates to novel devices and methods of use thereof. Specifically, the invention provides novel devices and methods for treating and/or promoting recovery of Anorectal and similar disorders.

### BACKGROUND OF THE INVENTION

Disorders of the lower Gastrointestinal tract affect millions each year all over the world. Frequently, these disorders dramatically alter the quality of life of a patient and require long (sometimes chronic) and painful treatment and recovery. Anorectal disorders, such as anal fistula and anal fissure, are the most common pathological conditions of the lower GI tract. One of the prominent characteristics of these disorders is a lesion formed on the lining of the intestines. For example, anal fistula is characterized by the formation of an abnormal tunnel under the skin connecting the anal canal and the skin near the anus. Most anal fistulas form in reaction to an anal gland that has developed a pus-filled infection (abscess), thus posing an immediate threat of systemic infection. Anal fissure is a condition where a small tear or crack develops in the lining of the anus. Both conditions cause pain, bleeding, complications, and result in long and painful recovery. Untreated anal fistula and fissure can become a breeding ground for bacteria, leading to infection. The constant exposure of the open wound to fecal matter increases the risk of bacterial contamination. Chronic anal fissures that are left untreated can lead to the development of anal fistula, fecal incontinence, anal stenosis, and psychological distress.

Most anal fistulas will require surgery to fix. While most anal fissures, might not need any treatment at all, beyond self-care might only need a prescription cream for temporary pain relief, fissures lasting several weeks it is considered as a chronic condition and requires more sophisticated treatment. Frequently, the treatment of anal fistula and chronic anal fissure involves various invasive surgical procedures that, depending on severity, might require an extended recovery period or even hospitalization. These procedures are often painful, and patients may experience complications such as bleeding, infection, and recurrence.

So far, no effective minimal to no invasive treatment of the anorectal conditions successfully entered marketplace and patients keep suffering from poor quality of life and complications. Therefore, there is an urgent and unmet need for less invasive, cost-effective, and safe treatment options for patients afflicted with disorders of the lower GI tract.

### SUMMARY OF THE INVENTION

It is a principal object of the present invention to provide a treatment solution that overcomes the drawbacks of the current treatment options and offers a safe, effective, and minimally invasive alternative for treating anorectal disorders.

According to some embodiments, the invention provides a device that can be easily inserted into the anal canal, where it acts as a sealant, preventing the leakage of fecal matter and other bodily fluids from the anal canal into the disordered area (lesion) thus facilitating healing process.

According to some embodiments, the invention provides a tubular sleeve designed to be positioned in the lumen of at least one hollow organ or a part thereof, wherein at least a portion of the tubular sleeve comprises a mesh, and, wherein the mesh allows anchoring of the tubular sleeve to the surface of the lumen of the hollow organ by becoming physically engaged with the surface of the lumen of the hollow organ.

According to some embodiments of the above tubular sleeve, the at least one hollow organ is part of the gastrointestinal tract (GI tract) or digestive tract.

According to some embodiments of the above tubular sleeve, the at least one hollow organ or a part thereof is selected from the group comprising an alimentary canal, hindgut, colon, rectum, and anal canal.

According to some embodiments of the above tubular sleeve, the surface of the lumen is the gastrointestinal wall or the rectal wall.

According to some embodiments of the above tubular sleeve, comprising the mesh at the proximal end and/or a distal end.

According to some embodiments of the above tubular sleeve, two or more portions of the tubular sleeve comprise the mesh.

According to some embodiments of the above tubular sleeve, the mesh is selected from the group comprising a knitted mesh, a braided mesh, a woven mesh, or any combination thereof.

According to some embodiments of the above tubular sleeve, the mesh is a surgical mesh or a biologic mesh (Biomesh).

According to some embodiments of the above tubular sleeve, the mesh is made of a material selected from the group consisting comprising of a synthetic material, a semi-synthetic material, a natural material, an animal-derived material, a polymer, and any combination thereof.

According to some embodiments of the above tubular sleeve, the material is selected from the group comprising Polyester (POL), Polyvinylidene fluoride (PVDF), Polyethylene terephthalate (PET), Polypropylene (PP), Polytetrafluorethylene (PTFE), Expanded PTFE (ePTFE), Cellulose, Oxidized Regenerated Cellulose (ORC), Polyglactin, Vicryl (polyglactin), Polyglecaprone, Polyethylene (PE), poly (glycolic acid) (PGA), poliglecaprone, Polylactic acid (PLA), Poly(lactic-co-glycolic acid) (PLGA), Carboxymethyl cellulose (CMC), Polydioxanone (PDO, PDS), Silk, Nylon, Collagen, any suitable material, and any combination thereof.

According to some embodiments of the above tubular sleeve, the Animal-derived material is selected from the group comprising porcine dermis, porcine small intestine submucosa, bovine dermis or pericardium, dermis or fascia lata of a cadaveric human, Collagen, any suitable material, and any combination thereof.

According to some embodiments of the above tubular sleeve, the material is characterized by being at least one of: an absorbable material, a non-absorbable material, an inert material, a material resistant to infection, a material resistant fistula formation, a non-carcinogenic material, a non-allergenic material, a hypersensitive material, a sterilizable material, an adjustable material, a material resistant to physical manipulation, a durable material, and any combination thereof.

According to some embodiments of the above tubular sleeve, the mesh is characterized by having a pore size type selected from: a mono-filament type, a duo filament type, and a multi filaments type.

According to some embodiments of the above tubular sleeve, the mesh further comprises a coating.

According to some embodiments of the above tubular sleeve, the coating comprises at least one compound selected from the group comprising cellulose, collagen, fatty acids, a phospholipid, a fiber, Laminins, and any combination thereof.

According to some embodiments of the above tubular sleeve, the outer surface of at least one segment of the tubular sleeve further comprises at least one structure, selected from the group comprising bumps, ridges, scaffolds, sponges, membranes, any suitable structure, and any combination thereof.

According to some embodiments of the above tubular sleeve, the at least one structure is made of a polymer, silicone, a phospholipid, a fiber, Collagen, Laminins, and any combination thereof.

According to some embodiments of the above tubular sleeve, the polymer is selected from the group comprising a natural polymer, a modified polymer, a synthetic polymer, and a semi-synthetic polymer.

The tubular sleeve according to some of the above, further comprising a chemical fastener and/or an adhesive.

According to some embodiments of the above tubular sleeve, the chemical fastener and/or adhesive comprises at least one clotting factor, at least one cross linker, at least one antihemorrhagic agent, or any combination thereof.

According to some embodiments of the above tubular sleeve, the chemical fastener and/or adhesive comprises at least one compound selected from the group comprising Fibrinogen (or Factor I), Thrombin (Factor IIa), Prothrombin (Factor II), Factor IV, Factor V, Factor VII, Factor VIII, Factor IX , Factor X, Factor XIII (fibrin stabilizing factor), Prothrombin complex concentrate (PCC or factor IX complex), prekallikrein (PK), high molecular weight kininogen (HK), and any combination thereof.

According to some embodiments of the above tubular sleeve, the chemical fastener and/or adhesive further comprises a compound selected from the group comprising Aprotinin, Fibronectin, pancreatic trypsin inhibitors (BPTI), Cell adhesion molecules (CAMS) , Phospholipids, Calcium (Ca²⁺), Calcium Chloride (CaCl₂), Calcium alginate, Oxidized cellulose (OC), and any combination thereof.

According to some embodiments of the above tubular sleeve, the adhesive and/or chemical fastener is selected from the group comprising fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

According to some embodiments of the above tubular sleeve, the adhesive and/or chemical fastener is bio-compatible, nontoxic, bioabsorbable or any combination thereof.

The tubular sleeve according to some of the above embodiments, further comprising an extraluminal segment.

According to some embodiments of the above tubular sleeve, the extraluminal section is positioned adjacent to the orifice of the hollow organ.

According to some embodiments of the above tubular sleeve, the orifice is the anus.

According to some embodiments of the above tubular sleeve, the extraluminal section is designed to expand radially away from the orifice.

According to some embodiments of the above tubular sleeve, the extraluminal section further comprises a plurality of semi connected segments.

According to some embodiments of the above tubular sleeve, the tubular sleeve is flexible, expandable, elastic, or any combination thereof.

The tubular sleeve according to some of the above embodiments, made of an impermeable material.

According to some embodiments of the above tubular sleeve, the impermeable material is impermeable to the body fluids and/or solids and/or body waste present and/or passing through the hollow organ.

According to some embodiments of the above tubular sleeve, the material is selected from the group comprising silicone, polyurethane, thermoplastic elastomer, rubber, vinyl chloride resin, Gortex, Per- and polyfluoroalkyl substances (PFAS or PFASs), polytetrafluoroethyline (PTFE), expanded PTFE (ePTFE), polyethelene, polypropylene and any combination thereof.

The tubular sleeve according to some of the above embodiments, further comprising a mechanical attachment mechanism and/or a sensor.

According to some embodiments of the above tubular sleeve, the mechanical attachment is selected from the group comprising ballons, barbs, any suitable attachments, and any combination thereof.

It is another object of the present invention to provide a method of placing a tubular sleeve in a hollow organ, a portion of the hollow organ, and/or a region adjacent to the hollow organ, for a predetermined period, comprising the steps of:
a. providing the tubular sleeve of any one of the above embodiments,
b. inserting the tubular sleeve into the lumen of the hollow organ,
c. positioning the at least a portion of the tubular sleeve comprising the mesh in a close proximity to the surface of the lumen to allow physical contact of the at least a portion of the tubular sleeve comprising the mesh with the surface of the lumen of the hollow organ, and
d. allowing anchoring of the at least a portion of the tubular sleeve comprising the mesh to the surface of the lumen of the hollow organ,
to thereby place the tubular sleeve in the hollow organ.

According to some embodiments of the above method of placing a tubular sleeve in a hollow organ, the hollow organ is part of the gastrointestinal tract (GI tract) or digestive tract.

According to some embodiments of the above method of placing a tubular sleeve in a hollow organ, the hollow organ is selected from the group comprising an alimentary canal, hindgut, colon, rectum, anal canal, and any combination thereof.

According to some embodiments of the above method of placing a tubular sleeve in a hollow organ, the surface of the lumen is gastrointestinal wall or rectal wall.

The method according to some of the above embodiments, further comprising a step of applying a chemical fastener and/or an adhesive to the mesh and/or the surface of the lumen.

According to some embodiments of the above method of placing a tubular sleeve in a hollow organ, the step of applying a chemical fastener and/or an adhesive precedes step a.

According to some embodiments of the above method of placing tubular sleeve in a hollow organ, the step of applying a chemical fastener and/or an adhesive precedes step d.

According to some embodiments of the above method of placing a tubular sleeve in a hollow organ, the chemical fastener and/or adhesive comprises at least one clotting factor, at least one cross linker, at least one antihemorrhagic agent, or any combination thereof.

According to some embodiments of the above method of placing a tubular sleeve in a hollow organ, the chemical fastener and/or adhesive comprises at least one compound selected from the group comprising Fibrinogen (or Factor I), Thrombin (Factor IIa) , Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), and any combination thereof.

According to some embodiments of the above method of placing a tubular sleeve in a hollow organ, the chemical fastener and/or adhesive further comprises a compound selected from the group comprising Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), and any combination thereof.

According to some embodiments of the above method of placing a tubular sleeve in a hollow organ, the adhesive and/or chemical fastener is selected from the group comprising fibrin glue, fibrin sealant, a GREG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

According to some embodiments of the above method of placing a tubular sleeve in a hollow organ, the at least one hollow organ, a portion of the hollow organ, and/or a region adjacent to the hollow organ comprises at least one lesion.

According to some embodiments of the above method of placing a tubular sleeve in a hollow organ, the tubular sleeve is designed to isolate the lesion from the content of at least one of: the lumen of the hollow organ, the portion of the hollow organ, and/or the region adjacent to the hollow organ, or a combination thereof.

According to some embodiments of the above method of placing a tubular sleeve in a hollow organ, the at least one lesion is selected from an anastomosis, a fissure, and a fistula.

The method according to some of the above embodiments of the method of placing a tubular sleeve in a hollow organ, further comprising the step of preparing the tubular sleeve and/or a portion of the tubular sleeve.

According to some embodiments of the above method of placing a tubular sleeve in a hollow organ, the step of preparing the tubular sleeve and/or a portion of the tubular sleeve precedes step b.

According to some embodiments of the above method of placing a tubular sleeve in a hollow organ, the predetermined period of time is between 1 day to 28 days.

It is another object of the present invention to provide a method of accelerating recovery of at least one lesion in the surface of the hollow organ, a portion of the hollow organ, a region adjacent to the hollow organ, or any combination thereof, by isolating the at least one lesion from the contents of the of the hollow organ, the portion of the hollow organ, the region adjacent to the hollow organ, or any combination thereof, the process comprising temporarily placing at least portion of the tubular sleeve of any of the above embodiments in the lumen.

The method of accelerating recovery according to some of the above embodiments, the placing at least a portion of the tubular sleeve in the lumen comprises the steps of:
a. providing the tubular sleeve of any of the above embodiments,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

The method of accelerating recovery according to some of the above embodiments, further comprising a step of applying a chemical fastener and/or an adhesive to the mesh and/or the surface of the lumen.

According to some embodiments of the above method the step of applying a chemical fastener and/or an adhesive precedes step a.

According to some embodiments of the above method the step of applying a chemical fastener and/or an adhesive precedes step d.

According to some embodiments of the above method the chemical fastener and/or adhesive comprises at least one clotting factor, at least one cross linker, at least one antihemorrhagic agent, or any combination thereof.

According to some embodiments of the above method the chemical fastener and/or adhesive comprises at least one compound selected from the group comprising Fibrinogen (or Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), and any combination thereof.

According to some embodiments of the above method the chemical fastener and/or adhesive further comprises a compound selected from the group comprising Aprotinin, Fibronectin, pancreatic trypsin inhibitors (BPTI), Cell adhesion molecules (CAMs), Phospholipids, Calcium (Ca²⁺) , Calcium Chloride (CaCl₂), Calcium alginate, Oxidized cellulose (OC), and any combination thereof.

According to some embodiments of the above method the adhesive and/or chemical fastener is selected from the group comprising fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

The method according to some of the above embodiments, further comprising a step of applying at least one therapeutic agent to the surface of the lumen.

According to some embodiments of the above method the step of applying to the surface of the lumen at least one therapeutic agent is repeated at least two times.

According to some embodiments of the above method the therapeutic is selected from the group comprising anti-inflammatory compounds, antibiotics, antivirals, coagulants, healing promoting factors and any combination thereof.

It is a another object of the present invention to provide a method of shielding at least one lesion in the surface of the hollow organ, a portion of the hollow organ, a region adjacent to the hollow organ, or any combination thereof from the contents of the of the hollow organ, the portion of the hollow organ, the region adjacent to the hollow organ, or any combination thereof, the process comprising placing at least a portion of the tubular sleeve of any of the above embodiments, in the lumen.

The method according to some of the above embodiments the placing at least a portion of the tubular sleeve in the lumen comprises the steps of:
a. providing the tubular sleeve according to any of the above embodiments,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

The method according to some of the above embodiments, further comprising a step of applying a chemical fastener and/or an adhesive to the mesh and/or the surface of the lumen.

According to some embodiments of the above method, the step of applying a chemical fastener and/or an adhesive precedes step a.

According to some embodiments of the above method, the step of applying a chemical fastener and/or an adhesive precedes step d.

According to some embodiments of the above method, the chemical fastener and/or adhesive comprises at least one clotting factor, at least one cross linker, at least one antihemorrhagic agent, or any combination thereof.

According to some embodiments of the above method, the chemical fastener and/or adhesive comprises at least one compound selected from the group comprising Fibrinogen (or Factor I), Thrombin (Factor IIa) , Prothrombin (Factor II ), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), and any combination thereof.

According to some embodiments of the above method, the chemical fastener and/or adhesive further comprises a compound selected from the group comprising Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), and any combination thereof.

According to some embodiments of the above method, the adhesive and/or chemical fastener is selected from the group comprising fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

It is another object of the present invention to provide a tubular sleeve of any of the above embodiments, for use in accelerating recovery of at least one lesion in the surface of the hollow organ, a portion of the hollow organ, a region adjacent to the hollow organ, or any combination thereof.

It is another object of the present invention to provide a tubular sleeve designed to be positioned in the lumen of a hollow organ, wherein at least one portion of the tubular sleeve comprises a mesh, and, wherein the mesh allows anchoring of the tubular sleeve to the side wall of the lumen of the hollow organ by becoming physically engaged with the tissue of the side wall of the lumen of the hollow organ.

### BRIEF DESCRIPTION OF THE DRAWINGS

FIG. 1: Illustrates the anatomy of the colon and shows longitudinal cross section of the anus.

FIG. 2: Illustrates the anatomy longitudinal cross section of the anus with a cross section of anorectal disorders, an anal fistula on the upper right image and an anal fissure on the lower right image.

FIG. 3: Illustrates the device inserted to the anus, showing longitudinal cross section of the anus with the device, fixed, according to one embodiment of the present invention.

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The present invention aims at addressing issues related to disorders associated with lower GI tract, such as anal fistulas and fissures, by providing a medical device that can be inserted into the rectum and positioned internally. The device creates a seal between the rectum and the skin, diverting feces away from the disordered area and allowing it to heal naturally, thereby avoiding the need for surgical intervention and preventing leakage.

According to some embodiments, the present invention provides a non-invasive device for the treatment of gastrointestinal or digestive issues.

According to some embodiments, the present invention provides a tubular sleeve designed to be positioned in the lumen of at least one hollow organ or a part thereof. In the context of the invention, the term ‘sleeve’ refers, without limitation, to an article configured to assume a cylinder and/or cylinder-like hollow structure upon positioning in the lumen of at least one hollow organ or a part thereof. According to some embodiments once positioned, the sleeve is designed to conduct human excreta. In the context of the invention, the term ‘sleeve’ can be referred to and/or used interchangeably with the terms “conduit”, “tube”, “pipe”, “channel”, “duct”, and “tunnel”.

As used herein, the term “hollow organ” refers, without limitation, to a body organ having an internal volume, lumen, and/or cavity. As used herein, the term ‘lumen’ refers, without limitation, to the internal volume or cavity of the hollow organ and/or a part of the hollow organ.

Typically, the hollow organ has a lumen and walls, while the tissue lining the walls of the lumen, that can also be referred to as the “surface” or “surface of the lumen”, is continuously exposed to the contents of the lumen. The internal volume or lumen can be continuously, intermittently and/or periodically filled with liquids, gases, solids, emulsions, dispersion, solutions, suspensions and or any combination thereof.

According to some embodiments, the hollow organ is part of the GI tract. In the context of the invention, the term ‘Gastrointestinal Tract’ (also referred to as the GI tract, digestive tract, alimentary canal) refers, without limitation, to at least part of the tract or passageway of the digestive system connects the mouth to the anus.

The term ‘wall’ refers to the structure forming the inner surface of the hollow organ. According to some embodiments, the surface of the lumen or wall is the gastrointestinal wall. In some embodiments, the mucous membrane or mucosa. In some embodiments of the present invention, the mucosa comprises epithelium, lamina propria, and muscularis mucosae.

According to some embodiments, the hollow organ is part of the lower GI tract. In the context of the invention, the term “Lower GI Tract” refers, without limitation, to part of the GI tract where an extensive reabsorption of water and salt occurs in the right/proximal colon and continues throughout. The Lower GI Tract begins at the cecum and includes the appendix (humans only), colon, rectum, anal channel, and anus. An alternative common name of the Lower GI Tract is “Large Intestine”. The primary function of the large intestine is to dehydrate and store fecal material. In the context of the invention, the terms “Fecal Matter”, “Fecal Material”, “Feces”, “Stool”, or “Excrement” are interchangeable with each other and refer, without limitation, to waste matter discharged from the large intestine through the anus during defecation. Fecal matter is considered as part of human excreta and is typically made up of 75 percent water and 25 percent solid matter.

According to some embodiments, at least a portion of the tubular sleeve comprises a mesh. In the context of the invention, the term “mesh” is meant to be understood as any woven material like a net, lattice, or webbing with spaces in it. According to some embodiments, the mesh is a knitted mesh, a braided mesh, and/or a woven mesh.

According to some embodiments, the mesh is a surgical mesh. In the context of the invention the term “surgical mesh” refers, without limitation, to a loosely woven mesh, which is used in surgery as either a permanent or temporary structural support for organs and other tissues. Surgical mesh can be made from both inorganic and biological materials and is used in a variety of surgeries.

According to some embodiments, the mesh is a biologic mesh (Biomesh). As used herein, the term “Biomesh” refers, without limitation, to a type of surgical mesh made from an organic biomaterial. A non-limiting list of organic biomaterial suitable for Biomesh includes porcine dermis, porcine small intestine submucosa, bovine dermis or pericardium, and the dermis or fascia lata of a cadaveric human.

According to embodiments, the non-limited list of materials the mesh is made of includes: a synthetic material, a semi-synthetic material, a natural material, an animal-derived material, a polymer, or any other suitable material and/or any combination thereof.

According to embodiments, the non-limited list of materials the mesh is made of includes: Polyester (POL), Polyvinylidene fluoride (PVDF), Polypropylene (PP), Polyethylene terephthalate (PET), Polytetrafluorethylene (PTFE), Expanded PTFE (ePTFE), Cellulose, Oxidized Regenerated Cellulose (ORC), Polyglactin, Vicryl (polyglactin), poly(glycolic acid) (PGA), Polyglecaprone, Polyethylene (PE), Polylactic acid (PLA), poliglecaprone, Polydioxanone (PDO, PDS), Poly(lactic-co-glycolic acid) (PLGA), Carboxymethyl cellulose (CMC), Silk, Nylon, Collagen, or any other suitable material and/or any combination thereof.

According to embodiments, the mesh is made of an animal-derived material. A non-limiting list animal-derived materials includes, without limitation, porcine dermis, porcine small intestine submucosa, bovine dermis or pericardium, dermis, or fascia lata of a cadaveric human, collagen, or any other suitable material and/or any combination thereof.

According to embodiments, the material the mesh is made of, is characterized, without limitation, by being at least one of: an absorbable material, a non-absorbable material, an inert material, a material resistant to infection, a material resistant fistula formation, a non-carcinogenic material, a non-allergenic material, a hypersensitive material, a sterilizable material, an adjustable material, a material resistant to physical manipulation, a durable material, a flexible material, an elastic material, a bactericidal material, or any other suitable material and/or any combination thereof.

As used herein, the term “absorbable” refers, without limitation, to a material that can naturally dissolve and be absorbed by the body over time. In some embodiments, the absorbable material is hydrolyzed or broken down by any of the adjacent cells, tissue or the content thereof, or of the hollow organ. In the context of the invention, the term “absorbable” can be referred to and/or used interchangeably with the terms “disintegratable”, “hydrolyzable”, “dissolvable”, and “degradable”.

In some embodiments of the present invention, the mesh is absorbed in between 1 day and 28 days. In some preferred embodiments the mesh is absorbed in between 3 and 21 days. According to some embodiments, the mesh is absorbed in between 7 to 18 days. In one embodiment, the mesh is absorbed in between 7 and 14 days.

According to some embodiments, the mesh is characterized by having a pore size type selected, without limitation, from a mono-filament type, a duo filament type, and/or a multi filaments type. In some embodiments, the mesh's Pore types may be, without limitation: macro-porous Type I; micro-porous Type II, macro-porous with multi-filamentous and/or micro porous components Type III; Biomaterials with sub-micrometer pore size Type IV.

According to some of the above embodiments, the mesh pore size depends on the material the mesh is made of.

According to some of the above embodiments, the pore size may be be ≥1000 μm for PP mesh, or ≥600 μm for PVDF mesh.

According to some of the above embodiments, orientation of the mesh may match the physiological stretchability of the tissue.

According to some of the above embodiments, the mesh comprises a coating. In some embodiments, the coating comprises, without limitation, at least of cellulose, collagen, fatty acids, phospholipids, fibers, laminins, or any other suitable material and/or any combination thereof.

According to some of the above embodiments, the mesh facilitates and/or enables anchoring of the tubular sleeve to the surface of the lumen of the hollow organ by becoming physically engaged with the surface of the lumen of the hollow organ.

According to some embodiments of the present invention, the mesh might be, without limitation, knitted mesh, braided mesh, woven mesh, and/or any combination thereof, or any other type of mesh, webbing or netting.

According to some embodiments, the mesh material is characterized, without limitation, as:
Inert to the content of the hollow organ and the wall or tissue of the hallow organ.
Having minimal adhesion formation.
Resistant to infection.
Resistant to fistula formation.
Having minimal pain and seroma formation.
Having excellent tissue ingrowth.
Having rapid engagement with/incorporation into the host tissue.
Non-carcinogenic.
Non-allergenic.

According to some embodiments, the mesh is characterized by having the following Bio-mechanical properties:
Ability to maintain adequate long-term tensile strength to prevent early recurrence.
Minimal shrinkage.
Anisotropy.
Elasticity.
Elongation
Capable of maintaining natural respiratory movements of the gastric wall.
Durable.

According to some embodiments, the mesh is characterized as:
Sterilizable.
Adjustable.
Easy to handle.

According to some embodiments, the mesh is positioned at the proximal end of the tubular sleeve. As used herein, the term “proximal end” refers, without limitation, to the sleeve end positioned or situated near the center of the body, and away from the opening.

According to some embodiments, at least two portions of the tubular sleeve of the invention comprise mesh. In some embodiments, mesh is positioned at the distal end of the sleeve.

The term “distal end” refers to the sleeve end farthest away from the center of the body and nearest to the opening of the hollow organ. In some embodiments, the distal end is extraluminal and positioned outside of the lumen of the hollow organ.

According to some embodiments of the present invention, two or more portions of the tubular sleeve comprise the mesh.

In some embodiments, the outer surface of at least one segment of the tubular sleeve further comprises at least one structure. Without limitation, the structure is selected from bumps, ridges, scaffolds, sponges, membranes, any combination thereof, or any other relevant structure.

According to some embodiments of the above tubular sleeve, the at least one structure is made of, without limitation, polymers, silicone, phospholipids, fibers, Collagen, Laminins, or any other suitable material and/or any combination thereof.

In some embodiments of the present invention, the polymer is selected from the non-limiting group comprising natural polymers, modified polymers, synthetic polymers, and semi-synthetic polymers.

According to some of the above embodiments, the tubular sleeve or mesh further comprises a chemical fastener and/or an adhesive.

The terms “adhesive” and “chemical fastener” refer, without limitation, to any material applied to at least one of two separate surfaces that binds or connects them together and resists their separation. The term adhesive can be used interchangeably with the term glue, cement, mucilage, and paste. In some embodiments, the adhesive or chemical fastener is characterized as a surgical adhesive, tissue adhesive or glue, or biological adhesive. In one embodiment, the adhesive or chemical fastener is characterized as a Pressure-sensitive adhesive (PSA).

According to some embodiments, tissue glues or biological adhesives can be characterized as natural protein-based (such as fibrin, thrombin, gelatine, albumin adhesives), synthetic glues (such as cyanoacrylate, polyethylene glycol, glutaraldehyde), biomimetic adhesives (glues secreted by lizards, mussels), and hybrid adhesives (which can be activated by light or temperature). The adhesive usually consists of monomers and/or polymers functionalized with reactive groups, e.g., acrylate nitrile, thiols, and others.

According to some embodiments, the chemical fastener and/or adhesive forms a plurality of blood clots (thrombi), of fibrin clots, a fibrin scaffold, a network of cross-linked fibrin proteins, or any combination thereof between the mesh and the tissue of the hollow organ. In some embodiments, the clots are formed in between, around, through, within and/or over fibers of the mesh, the pores of the mesh, or any combination thereof. In some embodiments, the network of cross-linked fibrin proteins or fibrin scaffold is interwoven with the mesh of the present invention.

According to some embodiments of the present invention, the chemical fastener and/or adhesive includes at least one clotting factor, at least one cross linker, at least one antihemorrhagic agent, or any other suitable material and/or any combination thereof.

According to some embodiments, the chemical fastener or adhesive includes at least two clotting factors.

According to some embodiments of the present invention, the chemical fastener and/or adhesive includes at least one compound selected from the non-limiting group including Fibrinogen (or Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), or any other suitable compound and/and any combination thereof.

According to some embodiments of the present invention, the chemical fastener and/or adhesive further comprises a compound selected from the non-limiting group comprising Aprotinin, Fibronectin, pancreatic trypsin inhibitors (BPTI), Cell adhesion molecules (CAMS), Phospholipids, Calcium (Ca²⁺), Calcium Chloride (CaCl₂), Calcium alginate, Oxidized cellulose (OC), or any other suitable compound and/and any combination thereof.

According to some embodiments, the adhesive and/or chemical fastener is selected from the group including fibrin glue, fibrin sealant, a cyanoacrylate glue, a GRFG (gelatin-resorcinol-formaldehyde/glutaraldehyde) glue, a polysaccharide adhesive, a polypeptide-based adhesive, a polymeric-based adhesive, a Polyethylene glycol (PEG) based adhesive, and matrix protein adhesives.

According to some embodiments of the present invention, the adhesive and/or chemical fastener is bio-compatible, nontoxic or non-cytotoxic, bioabsorbable or any combination thereof.

According to some embodiments of the present invention, the adhesive and/or chemical fastener is absorbed in between 1 day and 28 days. According to some embodiments, adhesive and/or chemical fastener is absorbed in between 3 and 21 days. According to some embodiments, adhesive and/or chemical fastener is absorbed in between 7 to 18 days. In one embodiment, adhesive and/or chemical fastener is absorbed in between 7 and 14 days.

According to some embodiments of the present invention, the tubular sleeve further comprises an extraluminal segment or portion. The term “extraluminal” refers to the segment of the sleeve found outside of the lumen of the hollow organ. In some embodiments of the present invention, the extraluminal section is positioned adjacent to the orifice of the hollow organ.

The term “orifice” (or body orifice) refers to any opening on the body of a human or an animal. In the context of the invention, the term “orifice” can be referred to and/or used interchangeably with the terms “opening”, and “hole”.

In some embodiments of the present invention, the orifice is the anus.

According to some embodiments of the present invention, the extraluminal section is designed or configured to expand radially away from the orifice.

In another embodiment of the present invention, the extraluminal section further comprises a plurality of semi connected segments.

According to some embodiments of the present invention, the extraluminal section allows anchoring of the tubular sleeve to the outer surface of the lumen of the hollow organ by becoming physically engaged with the surface of the lumen of the hollow organ. In one embodiment, the surface is the skin around the anal opening. According to one embodiment, the extraluminal section further comprises a chemical fastener and/or an adhesive.

According to some embodiments of the present invention, the tubular sleeve is flexible, expandable, elastic, or any combination thereof.

According to some embodiments, the tubular sleeve of the present invention is made of an impermeable material. In some embodiments of the present invention, the impermeable material is impermeable to the body fluids and/or solids and/or body waste present and/or passing through the hollow organ.

According to some of the above embodiments, the impermeable material can be, without limitation, silicone, polyurethane, thermoplastic elastomer, rubber, vinyl chloride resin, Gore-tex, Per- and polyfluoroalkyl substances (PFAS or PFASs), polytetrafluoroethyline (PTFE), expanded PTFE (ePTFE), polyethelene, polypropylene or any other suitable material and/or any combination thereof.

According to some embodiments of the present invention, the surface of at least one segment of the tubular sleeve further comprises a coating. In some embodiments of the present invention, the sleeve comprises a coating on the internal surface, the external surface, or both.

In one embodiment of the present invention, the coating comprises a therapeutic, or an active pharmaceutical ingredient (API). In one embodiment, the therapeutic is selected from the non-limiting group of anti-inflammatory compounds, antibiotics, antivirals, coagulant, and any other suitable compound and/or any combination thereof.

According to some embodiments of the present invention, the tubular sleeve further comprises a mechanical attachment mechanism and/or a sensor.

According to some embodiments, the mechanical attachment is selected from the non-limiting group including ballons, barbs, and any combination thereof.

In some embodiments of the present invention, the sleeve further comprises a fluid conduit for injecting a fluid into the hollow organ. According to some embodiments, the conduit injects the fluid into the space or volume formed between the outer surface of the tubular sleeve and the surface of the lumen. According to some embodiments, the fluid could be a solution, suspension, a dispersion, an emulsion, or any combination thereof. In some embodiments, the fluid comprises a therapeutic, and/or an active pharmaceutical ingredient (API). In some embodiments, the therapeutic is selected from the non-limiting group of anti-inflammatory compounds, antibiotics, antivirals, coagulant, and any combination thereof.

According to some embodiments of the present invention, the surface of at least one segment of the tubular sleeve or mesh is functionalized or treated. The surface can be functionalized or treated by drying, heating, sanitizing or any combination thereof.

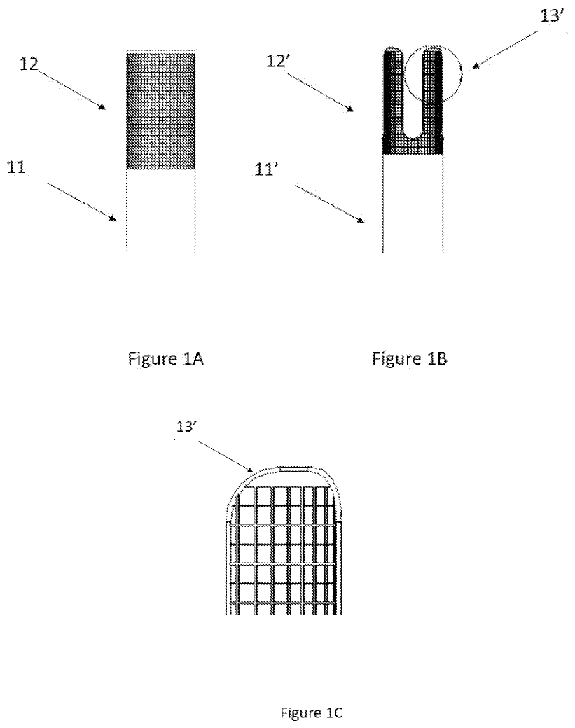

Reference is now made to FIG. 1A-C, presenting an exemplary embodiment of the tubular sleeve of the invention. FIG. 1A shows a proximal end of a tubular sleeve 11, comprising a mesh 12. FIG. 1B shows a tubular sleeve 11, comprising a mesh 12, with the mesh forming semi-connected sections. FIG. 1C shows a detailed view of mesh 13.

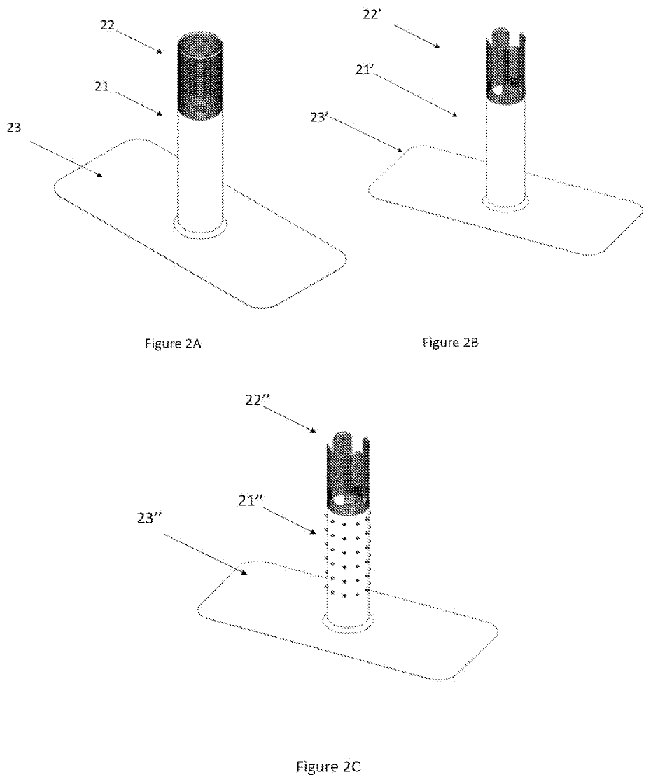

Reference is now made to FIG. 2A-C, presenting an exemplary embodiment of the tubular sleeve of the invention, configured to be placed in the anal canal. FIG. 2A shows an embodiment of the present invention, having a tubular sleeve 21, comprising a mesh 22, configured to be positioned in the gastric track, and an extraluminal section 23 configured to be positioned in the gastric track. FIG. 2B shows an embodiment of the present invention, having a tubular sleeve 21′, comprising a mesh with semi-connected sections 22′, and an extraluminal section 23′. FIG. 2C shows an embodiment of the present invention, having a tubular sleeve having bumps 21″, comprising a mesh with semi-connected sections 22″, and an extraluminal section 23″.

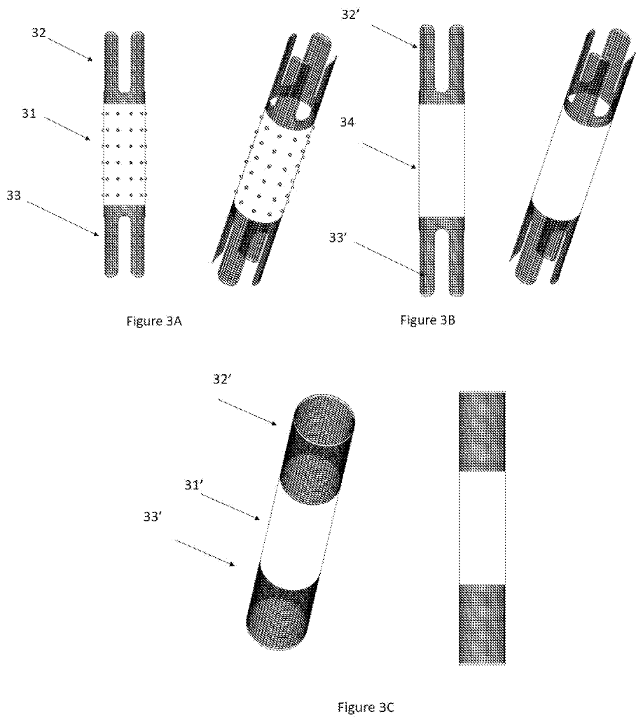

Reference is made to FIG. 3A-C, presenting an exemplary embodiment of the tubular sleeve of the invention, configured to be placed in the gastric track. FIG. 2A shows an embodiment of the present invention, having a tubular sleeve 21, comprising a mesh 22, and an extraluminal section 23. FIG. 2B shows an embodiment of the present invention, having a tubular sleeve 21′, comprising a mesh with semi-connected sections 22′, and an extraluminal section 23′. FIG. 2C shows an embodiment of the present invention, having a tubular sleeve having bumps 21″, comprising a mesh with semi-connected sections 22″, and an extraluminal section 23″.

According to some embodiments, the present invention provides a method for non-invasive treatment of gastrointestinal or digestive conditions or illnesses. As used herein, the term “condition” is meant to be understood as any impaired function or structure of the body, specifically of the gastric or digestive system the normal functioning of the body. According to some embodiments, the non-limiting list of conditions includes anorectal fistula, rectovaginal fistula treatment, an enterovaginal fistula, a urethral fistula, a vesicointestinal fistula, a gastrojejunocolic fistula, a gastrocolic fistula, an enterocutaneous fistula, a trachoesophageal fistula and a peptic ulcer disease.

According to some embodiments, the present invention provides a method of placing a tubular sleeve in a hollow organ, a portion of the hollow organ, and/or a region adjacent to the hollow organ, for a predetermined period of time.

According to some embodiments, the above method comprises the steps of:
a. providing the tubular sleeve according to any one of the above embodiments,
a. inserting the tubular sleeve into the lumen of the hollow organ,
b. positioning the at least a portion of the tubular sleeve comprising the mesh in a close proximity to the surface of the lumen to allow physical contact of the at least a portion of the tubular sleeve comprising the mesh with the surface of the lumen of the hollow organ, and
c. allowing anchoring of the at least a portion of the tubular sleeve comprising the mesh to the surface of the lumen of the hollow organ,
to thereby place the tubular sleeve in the hollow organ.

According to some embodiments of the above method, the hollow organ is part of the gastrointestinal tract (GI tract) or digestive tract.

According to some embodiments of the above method, the hollow organ is selected from, without limitation, the alimentary canal, hindgut, colon, rectum, anal canal, and any combination thereof.

According to some embodiments of the above method, the surface of the lumen is gastrointestinal wall or rectal wall.

According to some embodiments, the above method further comprises a step of applying a chemical fastener and/or an adhesive to the mesh and/or the surface of the lumen.

According to some embodiments of the above method, the step of applying a chemical fastener and/or an adhesive precedes step a.

According to some embodiments of the above method, the step of applying a chemical fastener and/or an adhesive precedes step d.

According to some embodiments of the above method, the chemical fastener and/or adhesive may be, without limitation, at least one clotting factor, at least one cross linker, at least one antihemorrhagic agent, or any other suitable material and/or any combination thereof.

According to some embodiments of the above method, the chemical fastener and/or adhesive comprises at least one compound selected from the non-limiting group including Fibrinogen (or Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), any other suitable material and any combination thereof.

According to some embodiments of the above method, the chemical fastener and/or adhesive further comprises a compound selected from the non-limiting group including Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), any other suitable material and/or any combination thereof.

According to some embodiments of the above method, the adhesive and/or chemical fastener is selected, without limitation, from fibrin glue, fibrin sealant, a GRFG (gelatin-resorcinol-formaldehyde/glutaraldehyde) glue, a cyanoacrylate glue, a polysaccharide adhesive, a polypeptide-based adhesive, polymeric-based adhesive, a Polyethylene glycol (PEG) based adhesive, and matrix protein adhesives.

According to some embodiments of the above method, the at least one hollow organ, a portion of the hollow organ, and/or a region adjacent to the hollow organ comprises at least one lesion.

According to some embodiments of the above method, the tubular sleeve is configured and/or designed to isolate the lesion from the content of at least one of: the lumen of the hollow organ, the portion of the hollow organ, and/or the region adjacent to the hollow organ, or a combination thereof. As used herein the term “to isolate” refers, without limitation, to the process of separating at least one portion of the lumen from its contents. In the context of the invention, the term “to isolate” can be referred to and/or used interchangeably with the terms “to shield”, “to separate” “to protect”, “to cover”, and “to block”.

According to some embodiments of the above method, the at least one lesion is selected, without limitation, from an anastomosis, a fissure, and a fistula. In some embodiments, the fistula could be characterized as an anorectal fistula, rectovaginal fistula treatment, an enterovaginal fistula, a urethral fistula, a vesicointestinal fistula, a gastrojejunocolic fistula, a gastrocolic fistula, an enterocutaneous fistula, a trachoesophageal fistula and a peptic ulcer disease. In another embodiments, the sleeve is configured to prevent leakage after coloanal anastomosis surgery or after rectal/anal perforation.

According to some embodiments, the above method further comprises a step of preparing the tubular sleeve and/or a portion of the tubular sleeve.

According some embodiments of the above method, the step of preparing the tubular sleeve and/or a portion of the tubular sleeve precedes step b.

According to some embodiments, the above method further comprises at least one step of functionalizing and/or treating the surface of at least one segment of the sleeve. The surface can be treated, without limitation, by drying, heating, sanitizing or any combination thereof.

According to some embodiments of the above method, the predetermined period of time is between 1 day and 28 days. In some preferred embodiments, the predetermined time is between 3 and 21 days. According to some embodiments, the predetermined time is 7 to 18 days. In one embodiment, the predetermined period of time is between 7 and 14 days.

According to some embodiments, the above method further comprises a step of positioning the extraluminal section. In one embodiment, the extraluminal section is positioned in close proximity to the surface of the body and adjacent to the orifice. In a preferred embodiment, the orifice is the anus.

According to some embodiments, the above method further comprises a step of anchoring the extraluminal section to the surface. In one embodiment, the step of anchoring the extraluminal section to the surface comprises the step of administering at least one adhesive.

According to some embodiments, the above method further comprises at least one step of functionalizing or treating the surface of at least one segment of the sleeve or the mesh. The surface can be treated, without limitation, by drying, heating, sanitating or any combination thereof.

According to some embodiments, the present invention provides a method of accelerating recovery of at least one lesion in the surface of the hollow organ, a portion of the hollow organ, a region adjacent to the hollow organ, or any combination thereof, by isolating the at least one lesion from the contents of the of the hollow organ, the portion of the hollow organ, the region adjacent to the hollow organ, or any combination thereof. According to some embodiments, the above method comprises temporarily placing at least a portion of the tubular sleeve, according to any of the above embodiments.

As used herein, the term “accelerating recovery” is interchangeably with terms “accelerating healing” and/or “promoting healing” and/or “improving” and or “delaying progression”, “slowing progression”, and refers, without limitation, to the positive effect the device of the invention might have on the clinical course of the pathological condition. For example, transformation from pathologic to non-pathologic state (full recovery); alleviation of the severity of symptoms of the pathological condition; reducing the time of transformation from pathologic to non-pathologic state; reduction of complications of the pathological condition (infection, injury and such). The “accelerating recovery” and/or “slowing progression” and/or “preventing progression” of the condition according to the embodiments of the above methods may be measured using any appropriate questionary, method, scale, diagnostic tool, or any other means that are known in the art or acceptable by the relevant functions and professionals. The term “preventing” might but does not necessarily mean recovery from the illness. As such, the term “preventing” relates to the situation when the patient does not present symptoms and/or signs and/or manifestations of the next “stage” of illness severity as defined by the appropriate and acceptable parameters for the specific pathological condition. The term “slowing”, or attenuating is can, without limitation, prolong the time of transition into the next “stage” of illness severity, thus providing greater window of opportunity for extensive care and recovery.

In some embodiments, the at least one lesion can be characterized, without limitation, as an anorectal fistula, rectovaginal fistula treatment, an enterovaginal fistula, a urethral fistula, a vesicointestinal fistula, a gastrojejunocolic fistula, a gastrocolic fistula, an enterocutaneous fistula, a trachoesophageal fistula and a peptic ulcer disease.

In some embodiments of the above method, the placing at least a portion of the tubular sleeve in the lumen comprises the steps of:
a. providing the tubular sleeve of any of the above embodiments,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

In some embodiments, the above method further comprises a step of applying a chemical fastener and/or an adhesive to the mesh and/or the surface of the lumen.

In one embodiment of the present method, the step of applying a chemical fastener and/or an adhesive precedes step a.

In another embodiment of the above method, the step of applying a chemical fastener and/or an adhesive precedes step d.

In some embodiments of the present method, the step of applying a chemical fastener and/or an adhesive further comprises a step of applying pressure.

According to some embodiments of the present method, the chemical fastener and/or adhesive includes at least one clotting factor, at least one cross linker, at least one antihemorrhagic agent, or any combination thereof.

In some embodiments of the present method, the chemical fastener and/or adhesive comprises, without limitation, at least one compound selected from the group of Fibrinogen (or Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), or any other suitable material and any combination thereof.

In some embodiments, the chemical fastener and/or adhesive further comprises a compound selected from the non-limiting group including Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), or any other suitable material and any combination thereof.

In some embodiments of the present method, the adhesive and/or chemical fastener is selected, without limitation, from the group of fibrin glue, fibrin sealant, a GREG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

According to some embodiments of the present method, the method further comprises a step of applying at least one therapeutic agent to the surface of the lumen.

In one embodiment of the above method, the step of applying to the surface of the lumen at least one therapeutic agent is repeated at least two times.

In some embodiments of the present method, the therapeutic is selected from the non-limiting group including anti-inflammatory compounds, antibiotics, antivirals, coagulants, healing promoting factors and any combination thereof.

In another embodiment, the method further comprises a step of positioning an extraluminal section. In one embodiment, the extraluminal section is positioned in close proximity to the surface of the body and adjacent to the orifice. In a preferred embodiment, the orifice is the anus. In one embodiment, the surface is the skin around the anal opening.

According to one embodiment, the method further comprises a step of anchoring the extraluminal section to the surface. In one embodiment, the step of anchoring the extraluminal section to the surface comprises the step of administering at least one adhesive.

In some embodiments of the present invention, the method further comprises at least one step of functionalizing or treating the surface of at least one segment of the sleeve. The surface can be treated by drying, heating, sanitizing or any combination thereof.

According to some embodiments, the present invention provides a method of shielding at least one lesion in the surface of the hollow organ, a portion of the hollow organ, a region adjacent to the hollow organ, or any combination thereof from the contents of the of the hollow organ, the portion of the hollow organ, the region adjacent to the hollow organ, or any combination thereof. According to some embodiments, the method comprises the step of placing at least a portion of the tubular sleeve of any of the above embodiments in the hollow organ.

In some embodiments, the at least one lesion can be characterized, without limitation, as an anorectal fistula, rectovaginal fistula treatment, an enterovaginal fistula, a urethral fistula, a vesicointestinal fistula, a gastrojejunocolic fistula, a gastrocolic fistula, an enterocutaneous fistula, a trachoesophageal fistula and a peptic ulcer disease.

In some embodiments of the present method, the step of the placing at least a portion of the tubular sleeve in the lumen comprises the steps of:
a. providing the tubular sleeve of any of the above embodiments,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

In some embodiments of the present method, the method further comprises a step of applying a chemical fastener and/or an adhesive to the mesh and/or the surface of the lumen.

According to some embodiments of the above method, the step of applying a chemical fastener and/or an adhesive precedes step a.

In another embodiment of the above method, the step of applying a chemical fastener and/or an adhesive precedes step d.

In some embodiments of the above method, the step of applying a chemical fastener and/or an adhesive further comprises a step of applying pressure.

According to some embodiments of the above method, the chemical fastener and/or adhesive comprises, without limitation, at least one clotting factor, at least one cross linker, at least one antihemorrhagic agent, or any other suitable material, or any combination thereof.

According to some embodiments of the above method, the chemical fastener and/or adhesive comprises at least one compound selected from the non-limiting group including Fibrinogen (or Factor I), Thrombin (Factor IIa), Prothrombin (Factor II), Factor IV, Factor V, Factor VII, Factor VIII, Factor IX, Factor X, Factor XIII (fibrin stabilizing factor) , Prothrombin complex concentrate (PCC or factor IX complex), prekallikrein (PK), high molecular weight kininogen (HK), or any other suitable material, and any combination thereof.

According to some embodiments of the above method, the chemical fastener and/or adhesive further comprises a compound selected from the non-limiting group including Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), or any other suitable material, and any combination thereof.

According to some embodiments of the above method, the adhesive and/or chemical fastener is selected, without limitation, fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

In another embodiment, the method further comprises a step of positioning and/or anchoring an extraluminal section. In one embodiment, the extraluminal section is positioned in close proximity to the surface of the body and adjacent to the orifice. In a preferred embodiment, the orifice is the anus. In one embodiment, the surface is the skin around the anal opening.

According to some embodiments of the above method, the extraluminal section s anchored to the surface. In one embodiment, the extraluminal section is anchored by applying or administering an adhesive or chemical fastener.

According to some embodiments of the above method, the step of applying or administering an adhesive or a chemical fastener, further comprises a step of applying pressure. In some embodiments of the present invention, the method further comprises at least one step of functionalizing or treating the surface of at least one segment of the sleeve. The surface can be treated by drying, heating, sanitizing or any combination thereof.

According to some embodiments, the present invention provides the tubular sleeve according to any of the above embodiments, for use in accelerating recovery of at least one lesion in the surface of the hollow organ, a portion of the hollow organ, a region adjacent to the hollow organ, or any combination thereof.

According to an embodiment, the present invention provides a method of preventing leakage from an orifice of an/a hollow organ, a portion of the hollow organ, an orifice, or any combination thereof from the contents of the of the hollow organ, the portion of the hollow organ, the region adjacent to the hollow organ, or any combination thereof. According to some embodiments, the method comprises the step of placing at least a portion of the tubular sleeve of any of the above embodiments in the hallow organ.

According to one embodiment of the above method, the sleeve is configured to prevent leakage following coloanal anastomosis surgery or after rectal/anal perforation.

According to some embodiments of the above method, the step of the placing at least a portion of the tubular sleeve in the lumen comprises the steps of:
a. providing the tubular sleeve of any of the above embodiments,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

In some embodiments of the present invention, the method further comprises a step of applying a chemical fastener and/or an adhesive to the mesh and/or the surface of the lumen.

In one embodiment of the present method, the step of applying a chemical fastener and/or an adhesive precedes step a.

In another embodiment of the above method, the step of applying a chemical fastener and/or an adhesive precedes step d.

In some embodiments of the above method, the step of applying a chemical fastener and/or an adhesive further comprises a step of applying pressure.

According to some embodiments of the above method, the chemical fastener and/or adhesive comprises, without limitation, at least one clotting factor, at least one cross linker, at least one antihemorrhagic agent, or any other suitable compound, or any combination thereof.

According to some embodiments of the above method, the chemical fastener and/or adhesive comprises at least one compound selected from the non-limiting group including Fibrinogen (or Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), or any other suitable compound, and any combination thereof.

According to some embodiments of the above method, the chemical fastener and/or adhesive further comprises a compound selected from the non-limiting group of Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), or any other suitable compound, and any combination thereof.

According to some embodiments of the above method, the adhesive and/or chemical fastener is selected, without limitation, from fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

In another embodiment, the method further comprises a step of positioning an extraluminal section. In one embodiment of the above method, the extraluminal section is positioned in close proximity to the surface of the body and adjacent to the orifice. In a preferred embodiment of the above method, the orifice is the anus. In another embodiment of the above method, the surface is the skin around the anal opening.

According to some embodiments of the above method, the extraluminal section is anchored to the surface. In a preferred embodiment, the extraluminal section is anchored by applying or administering an adhesive or chemical fastener.

According to some embodiments of the above method, the step of applying or administering an adhesive or a chemical fastener, further comprises a step of applying pressure.

According to some embodiments of the above method, the method further includes at least one step of functionalizing or treating the surface of at least one segment of the sleeve. The surface can be treated by drying, heating, sanitizing or any combination thereof.

### EXAMPLES

In the examples below, if an abbreviation is not defined above, it has its generally accepted meaning. Further,

### Example 1: Anchoring a Device in a Hollow Organ

To examine the suitability of the device of invention to allow anchoring of the mesh to the tissue the following steps are taken in-vitro:
1. The surface of the lumen is cleaned.
2. The components (Fibrin glue components, including Fibrinogen, thrombin and calcium salts, a tubular silicon steeve comprising a VKML-Vicryl (polyglactin) mesh, and any other materials) are checked for integrity.
3. The sleeve and mesh are cleaned and dried.
4. The fibrin glue components, including fibrinogen, thrombin, and calcium salts are mixed.
5. The mixed fibrin glue components, including Fibrinogen, thrombin, and calcium salts, are applied to the mesh.
6. The mesh, comprising the mixed fibrin glue components, is applied to the surface of the lumen.
7. Pressure is applied to join the mesh and the surface of the lumen.
8. The placement of the mesh and the adhesive strength are tested.

### Results

Surprisingly, the mesh is found suitable for anchoring the device to the tissue.

### Example 2: Placing the Device of the Invention in a Patient

To examine compatibility of the device of the invention, the following steps of placing the device into the patient are taken:
1. The patient's gastric track is emptied of its contents.
2. The Fibrin glue components, tubular silicon steeve comprising a VKML-Vicryl (polyglactin) mesh in the proximal end, and any other materials are checked for integrity.
3. The sleeve and mesh are cleaned and dried.
4. The fibrin glue components, fibrinogen, thrombin, and calcium salts are mixed.
5. The mixed fibrin glue components, including fibrinogen, thrombin, and calcium salts, are applied to the mesh.
6. The proximal end of the sleeve was placed in the anal canal.
7. The mesh, comprising the mixed fibrin glue components, is applied to the wall of the anal canal.
8. Pressure is applied to join the mesh and the wall of the anal canal.
9. The placement of the proximal end of the sleeve and the adhesive strength are tested.
10. The extraluminal section and surface surrounding the anus are cleaned and dried.
11. An adhesive is applied to the extraluminal section of the distal end.
12. The extraluminal section of the sleeve is pressed to the skin surrounding the anus.
13. The adhesive strength is tested.

### Results

Surprisingly, the device of the invention can be successfully placed in the anal canal of the patient and can be securely anchored.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will prevail. In addition, the materials, methods, and examples are illustrative only and are not intended to be necessarily limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

As used herein the terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise. For example, the term “a compound” or “at least one compound” may include a plurality of compounds, including mixtures thereof.

It will be understood that when an element is referred to as being “on,” “attached” to, “connected” to, “coupled” with, “contacting,” “engaged” with, etc., another element, it can be directly on, attached to, connected to, coupled with and/or contacting the other element or intervening elements can also be present. In contrast, when an element is referred to as being, for example, “directly on,” “directly attached” to, “physically engaged” with, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application various publications, published patent applications and published patents are referenced. The disclosures of these publications in their entireties are hereby incorporated by reference into this application in order to more fully describe the state of the art to which this invention pertains.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein the term “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

“Treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms, or relieving the disease, i.e., causing regression of the disease or its clinical symptoms.

A “therapeutically-effective amount” or an “effective amount” means the amount of a compound or a dosage form that, when administered to a subject for treating a disease, is sufficient to effect such treatment for the disease. The “therapeutically-effective amount” will vary depending on the compound, the disease, and its severity and the age, weight, etc., of the subject to be treated.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

1.-75. (canceled)

76. A tubular sleeve designed to be positioned in the lumen of at least one hollow organ or a part thereof, wherein at least a portion of the tubular sleeve comprises a mesh, and wherein the mesh allows anchoring of the tubular sleeve to the surface of the lumen of the hollow organ by becoming physically engaged with the surface of the lumen of the hollow organ.

77. The tubular sleeve of claim 76, wherein the at least one hollow organ is part of the gastrointestinal tract (GI tract) or a digestive tract and/or wherein the surface of the lumen is the gastrointestinal wall or the rectal wall.

78. The tubular sleeve of claim 76, wherein the mesh is characterized by at least one of the following:
a. the mesh is positioned at the proximal end and/or a distal end;
b. the mesh is selected from the group consisting of a knitted mesh, a braided mesh, a woven mesh, or any combination thereof;
c. the mesh made of a surgical mesh or a biologic mesh (Biomesh);
d. the mesh is made of a material selected from the group consisting of a synthetic material, a semi-synthetic material, a natural material, an animal-derived material, a polymer, and any combination thereof;
e. the mesh is made of a material selected from the group consisting of Polyester (POL), Polyvinylidene fluoride (PVDF), Polyethylene terephthalate (PET), Polypropylene (PP), Polytetrafluorethylene (PTFE), Expanded PTFE (ePTFE), Cellulose, Oxidized Regenerated Cellulose (ORC), Polyglactin, Vicryl (polyglactin), Polyglecaprone, Polyethylene (PE), poly(glycolic acid) (PGA), poliglecaprone, Polylactic acid (PLA), Poly(lactic-co-glycolic acid) (PLGA), Carboxymethyl Cellulose (CMC), Polydioxanone (PDO, PDS), Silk, Nylon, Collagen, and any combination thereof;
f. the mesh is made of an Animal-derived material selected from the group consisting of porcine dermis, porcine small intestine submucosa, bovine dermis or pericardium, dermis or fascia lata of a cadaveric human, Collagen, and any combination thereof;
g. the mesh made of a material characterized by being at least one of: an absorbable material, a non-absorbable material, an inert material, a material resistant to infection, a material resistant fistula formation, a non-carcinogenic material, a non-allergenic material, a hypersensitive material, a sterilizable material, an adjustable material, a material resistant to physical manipulation, a durable material, and any combination thereof;
h. the mesh made of a material characterized by having a pore size type selected from: a mono-filament type, a duo filament type, and a multi filaments type; or,
i. the mesh further comprises a coating.

79. The tubular sleeve of claim 78, wherein the coating comprises at least one compound selected from the group consisting of cellulose, collagen, fatty acids, a phospholipid, a fiber, Laminins, and any combination thereof.

80. The tubular sleeve of claim 76, wherein the outer surface of at least one segment of the tubular sleeve further comprises at least one structure, wherein the structure is selected from the group consisting of bumps, ridges, scaffolds, sponges, membranes, and any combination thereof, and/or wherein the structure is made of polymers, silicone, phospholipids, fibers, Collagen, Laminins, or any combination thereof.

81. The tubular sleeve of claim 76, further comprising a chemical fastener and/or an adhesive.

82. The tubular sleeve of claim 81, wherein the chemical fastener and/or adhesive is characterized by at least one of the following:
a. comprising at least one clotting factor, at least one cross linker, at least one antihemorrhagic agent, or any combination thereof;
b. comprising at least one compound selected from the group consisting of Fibrinogen (Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), and any combination thereof;
c. comprising a compound selected from the group consisting of Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), and any combination thereof;
d. selected from the group consisting of a fibrin glue, a fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives; or
e. bio-compatible, nontoxic, bioabsorbable or any combination thereof.

83. The tubular sleeve of claim 76, wherein the tubular sleeve is characterized by at least one of the following:
a. comprising an extraluminal segment;
b. as being flexible, expandable, elastic, or any combination thereof; or, c. as being made of an impermeable material.

84. The tubular sleeve of claim 83, wherein the extraluminal section is positioned adjacent to the orifice of the hollow organ, is designed to expand radially away from the orifice, comprises a plurality of semi connected segments, or any combination thereof.

85. A method of accelerating recovery of at least one lesion in the surface of the hollow organ, a portion of the hollow organ, a region adjacent to the hollow organ, or any combination thereof, by isolating the at least one lesion from the contents of the of the hollow organ, the portion of the hollow organ, the region adjacent to the hollow organ, or any combination thereof, the process comprising temporarily placing at least a portion of the tubular sleeve of claim 76 in the lumen.

86. The method of claim 85, wherein the placing at least a portion of the tubular sleeve in the lumen comprises the steps of:
a. providing the tubular sleeve,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

87. The method of claim 86, further comprising at least one step selected from:
a. applying a chemical fastener and/or an adhesive to the mesh and/or the surface of the lumen; and,
b. applying at least one therapeutic agent to the surface of the lumen.

88. The method of claim 87, wherein the chemical fastener and/or adhesive is characterized by at least one of the following:
a. comprising at least one clotting factor, at least one cross linker, at least one antihemorrhagic agent, or any combination thereof;
b. comprising at least one compound selected from the group consisting of Fibrinogen (Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), and any combination thereof;
c. comprising a compound selected from the group consisting of Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), and any combination thereof; or,
d. is selected from the group consisting of fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.

89. The method of claim 87, wherein the therapeutic is selected from the group comprising anti-inflammatory compounds, antibiotics, antivirals, coagulants, healing promoting factors and any combination thereof.

90. A method of shielding at least one lesion in the surface of the hollow organ, a portion of the hollow organ, a region adjacent to the hollow organ, or any combination thereof from the contents of the of the hollow organ, the portion of the hollow organ, the region adjacent to the hollow organ, or any combination thereof, the process comprising placing at least a portion of the tubular sleeve of claim 76 in the lumen.

91. The method of claim 90, wherein the placing at least a portion of the tubular sleeve in the lumen comprises the steps of:
a. providing the tubular sleeve,
b. inserting the tubular sleeve into the lumen,
c. positioning the at least a portion of the tubular sleeve comprising the mesh in a close proximity to the surface of the lumen to allow physical contact of the at least a portion of the tubular sleeve comprising the mesh with the surface of the lumen, and
d. allowing anchoring of the at least a portion of the tubular sleeve comprising the mesh to the surface of the lumen.

92. The method of claim 90, further comprising a step of applying a chemical fastener and/or an adhesive to the mesh and/or the surface of the lumen.

93. The method of claim 92, wherein the chemical fastener and/or adhesive is characterized by at least one of the following:
a. comprising at least one clotting factor, at least one cross linker, at least one antihemorrhagic agent, or any combination thereof;
b. comprising at least one compound selected from the group consisting of Fibrinogen (Factor I), Thrombin (Factor IIa), Prothrombin (Factor II), Factor IV, Factor V, Factor VII, Factor VIII, Factor IX, Factor X, Factor XIII (fibrin stabilizing factor), Prothrombin complex concentrate (PCC or factor IX complex), prekallikrein (PK), high molecular weight kininogen (HK), and any combination thereof;
c. comprising a compound selected from the group consisting of Aprotinin, Fibronectin, pancreatic trypsin inhibitors (BPTI), Cell adhesion molecules (CAMs), Phospholipids, Calcium (Ca²⁺), Calcium Chloride (CaCl₂), Calcium alginate, Oxidized cellulose (OC), and any combination thereof; or,
d. is selected from the group consisting of fibrin glue, fibrin sealant, a GRFG (gelatin-resorcinol-formaldehyde/glutaraldehyde) glue, a cyanoacrylate glue, a polysaccharide adhesive, a polypeptide-based adhesive, a polymeric-based adhesive, a Polyethylene glycol (PEG) based adhesive, and matrix protein adhesives.
